SEQUENCE LISTING (23 sequences)

SEQ ID NO: 1 - DNA, 23 bp, Artificial
  oligonucleotide primer that amplifies mouse anti-human monoclonal antibody cDNA
gcatgcgcgcggccgcggaggcc

SEQ ID NO: 2 - DNA, 35 bp, Artificial
  oligonucleotide primer that amplifies mouse anti-human monoclonal antibody cDNA
gcatgcgcgcggccgcggaggcccccccccccccc

SEQ ID NO: 3 - DNA, 48 bp, Artificial
  oligonucleotide primer that amplifies mouse anti-human monoclonal antibody cDNA
ctctaagcttggctcaaacacagcgacctcggatacagttggtgcagc

SEQ ID NO: 4 - DNA, 45 bp, Artificial
  oligonucleotide primer that amplifies mouse anti-human monoclonal antibody cDNA
ctcttctagagagtctctcagctggtaggatacagttggtgcagc

SEQ ID NO: 5 - DNA, 357 bp, Artificial
  mouse anti-human monoclonal antibody cDNA
gacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctca

SEQ ID NO: 6 - protein, 119 aa, Artificial
  mouse anti-human monoclonal antibody
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 7 - DNA, 321 bp, Artificial
  mouse anti-human monoclonal antibody cDNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaa

SEQ ID NO: 8 - protein, 107 aa, Artificial
  mouse anti-human monoclonal antibody
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIK

SEQ ID NO: 9 - DNA, 2431 bp, Artificial
  mouse anti-human monoclonal antibody cDNA
tcatgacattaacctataaaaataggcgtatcacgaggccctttcgtcttcaagaattcttcagatacaaagaatctctaaaccctgaggacattctatcacaaataagtaaaattcagaaaattctgaatgctcccatcacagagatgaatctgctatgaacagctcataggtgtgaagctctacaaaagccatattattgaaaagccacattgtgcccagactttggaaagactgagctcatatcctgaaatacagttatgtgtggttctatctaattacacatttacactaaggaaacatggcagtatgggaatgaagcttgttctgtacacattaacagagggaaactaaacaaagtatggtgaatccctaaccaaaagtaaaaaaaaaaaaaaaaaagaaaagaaaagaaaaaaaaagtgaaactacaatatgtttcaaatgctgtaactgaaatctggttttttgatgccttatatctgttatcatcagtgacttcagatttagtccaactccagagcatggtatagcaggaagacatgcaaataggtcttctctgtgcccatgaaaaacacctcggccctgaccctgcagctctgacagaggaggcctgtcctggattcgattcccagttcctcacattcagtgatcagcactgaacacagacccctcaccatgaacttcgggctcagattgattttccttgtcctggttttaaaaggtatcttattgagtatagaggacatctgctgtatgcacagaggtgcagaaaaaatgttgtttgttttttttagtgacaatgctccaaacagtattctttctttgcaggtgtcctgtgtgacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctcaggtaagaatggcctctccaggtcttttttttaatctttgtaatggagttttctgaacattgcagactaatcttggatatttgtccctgaggtagccggctgagagaaattgggaattaaactgtctcgggatctcagagcctttaggacagattatctccacatctttgaaaaactgagattctgtgtgatggtgttggtggagtccctggatgatgggatagggactttggaggctcatttgagggagatgctaaaacaatcctatggctggagggagagttggggctgtagttggagattttcagtttttagaataaaagctttagctgcgggaaatccttcaggaccacctctgtgacagcatttatacagtatccgatgcatagggacaaagagtggagtggggcactttctttcgatttgtggggaatgttccacactagtttctgtgaaacctcatttgttggagggagagctgtcttagtgcctgagtcaagggagaagggcatctagcctcggtctcaaaagggtagttgctgtccagagaggtctggtggagcctgcaaaagtccagctttcaaaggaacacagaagtatgtgtatggaataatagaagatgttgcttttactcttaagttggttcataggaaaaatagttaaaactgtgagtttaaaatgtgagagggttttcaagtactcatttttttacatgtccaaaatttctgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcatt

SEQ ID NO: 10 - DNA, 5557 bp, Artificial
  mouse anti-human monoclonal antibody cDNA
aattccaagctttgtatcttcagatccaggaaagccaccaccaatatcaaacagatacatgctgaaaccaacttctgttcttatgtcaaatgcacagcgggcatctgacactgcctgcatgaaggtctcaggtcaatacttccactacacacatggaagctgacaccaatgacgtcaatatttagctcttttgcccatttcaggaggagactgctggttttgagtgtggcaccagacttaacaccaagtcgacaaactgctttggaatcatctgtgacaatccacaaaaacaactttgtcttacaatgtgctctgacgacattcatcaattcatttcactgtcaaaagtcatcatctggactccattactggcagcatacttgatttgagacacttgtttacaaaaatgtgcataggtaatcctctctggaggaaccagaagcccccgttccaactgtatttcagtcttgcttgcacagtcaaatcctgtaccaatagcagctagggtgttaactatggctctgttgtccttacacttgactgcacaaaaaggaataacattcggaagagcttttagccacctcagatgcttctttagaatgtctctgaggtccggaacctagaaagaagagacttcatttattattttgtgttcagaatgtccttagcactaaagccaccatctatgatacagcagtcaaactcttccttagtatagctgctcatcgttctccatgtgcctacagaaaacctagacatggaattaaattattgccagccccttacaaggtcaacttatccaagaactgtgaatgcagactccttgaaatgttggaaacactcacagcacagggtcaagactggctggacacatggagacactgaatcctgaagagcacttagctgtctgttgcttcatcatgtctactgacctgaggtggcaccaagctgcttactgagggaggactgtggcggtgtctgcaggaactgacaattctccacaattctcttactgccccactcataactcttctcttctccatcttcttctttctttcctctcccctcctttttccctttcactacttttttcctttcttcttttccacttcccttttctttcttcttttgctgttgctgttgtaaaggatttattgtttcctcgtgattgaaccaaaggtagttgtactattatttctgtaaaactcatctgttgattttctattaattaattaattttgtttacactccatattttattcaacccctccatcctcctactggtctacataccatacctccttcccacacccctgtctccacatggatgctgccacctcccatgccacctgacctctcatctccctagggcatctagtctcttgaggcttagatgcatcatttctgagtgaacacagatccaacaatcctctgctatatgtgtgttggtggcctcatagcagctggtgtatgctgcctgtttgttgatccagtgtttgagaggtctcgcgggttcagattaattgagattgttggacctcctcagcgtctttcagtctttccctgattcaacaacagggttcattgtttctgttcattggttgggtgcaaatatctgcatctgactcagctgcttattgggtcttctggagtgcagtcatgctaggtccgtttctatgagtgctccatagcctcagtgatagtgtcaggcgttgggactgccccttgacctggattctattttggacctgtcgctggaccttcttttcctcaggctcccctccatctgtatccctgtaattctttcagacaggaacaaatatgggtcagagttgtgagtgtggaatggcacccccttccctcatttaatgccctgtcttcctggtggaagtgggctctataagttcccactccctactgttgggcatttcatccctttgagtcctgagagtctctcacctcccaggtctctggtgcattctggagggtcctcccaacctcctacctccccaggttgcctgttgacagacttctgctggcccccagtgcttcagtccttttccctcacccaatatctgatttggatggaagcctgtcatgagaacatctatatacttgtggtttcagagctttaaattggtccttgagcttctattttgagttcctttccagtgattacttgctgtctttggtagtacttttgactgtttatttaacctggatactctcatacagctgtgtaatttacttccttatttgatgactgctttgcatagatccctagaggccagcccagctgcccatgatttataaaccaggtctttgcagtgagatctgaaatacatcagaacagcatgggcttcaagatggagtttcatactcaggtctttgtattcgtgtttctctggttgtctggtgagaattttaaaagtattataacatctcaaaagtaatttatttaaatagcttttcctataggaagccaatattaggcagacaatgccattagataagacattttggattctaacatttgtgtcaaaaatctttgtatatataagtgtttactcattatctatttctgattgcaggtgttgatggagacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaacgtaaatagaatccaaactctctttcttccgttgtctatgtctgtggcttctatgtctaaaaatgatgtagatattttttctctgagaccagattctgtcactctccaaggcaaagatacatagtcactccgtaagcagagctgggaataggctagacatgttctctggagaatgaatgccagtgtaataattaacacaagtgatagtttcagaaatgctcaaagaagcagggtagcctgccctagacaaacctttacttggtgctcagaccatgctcagtttttgtatgggggttgagtgaagggacaccagtgtgtgtatacgttcggaggggggaccaagctggaaataaaacgtaagttgtcttctcaactcttgttcactgagtctaaccttgttactttgttctttgttgtgtgtttttcttaaggagatttcagggatgtatcaaattccattctcagatcaggtgttaaggagggaaaacttgtcccacaagaggttggaatgattttcaggctaaattttaggcttctaaaccaaagtcattaaactaggggaagagggataattgtctgcctagggagggttttgtggaagtacagttaaagtagatcactgtaaaccacattcagagatgggaccagactggaaataaaacctaagaacatttttgctcaactgcttgtgaagttttggtcccattgtgtcctttgtgtgagtttgtggtgttcattagataaatgaactattccttgtaacccaaaacttaaatagacgagaaccaaaaatctagctactgtataagttgagcaaacagactgacctcatgtcagatttgtgggagaaatgagaaaggaacagtttttctctgaacttggcctatctaactggatcagcctcaggcaggtttttgtaaaggggggcacagtgatatgaatcactgtgattcacgttcggctcggggacaaagttggaaataaaacgtaagtagatttttgctcatttacttgtgacgttttggttctgtttgggtaactcgtgtgaatttgtgacattttggctaaatgagccattcctggcaacctgtgcatcaatagaagatcccccagaaaagagtcagtgtgaaagctgagcgaaaaactcgtcttaggcttctgagaccagttttgtaaggggaatgtagaagaaagagctgggcttttcctctgaatttggcccatctagttggactggcttcacaggcaggtttttgtagagaggggcatgtcatagtcctcactgtggctcacgttcggtgctgggaccaagctggagctgaaacgtaagtacacttttctcatctttttttatgtgtaagacacaggttttcatgttaggagttaaagtcagttcagaaaatcttgagaaaatggagagggctcattatcagttgacgtggcatacagtgtcagattttctgtttatcaagctagtgagattaggggcaaaaagaggctttagttgagaggaaagtaattaatactatggtcaccatccaagagattggaccggagaataagcatgagtagttattgagatctgggtctgactgcaggtagcgtggtcttctagacgtttaagtgggagatttgggggggatgaggaatgaaggaacttcaggatagaaaaggtctgaagtcaagttcagctcctaaaatggatgtgggagcaaactttgaagataaactgaatgacccagaggatgaaacagtgcagatcaaagaggggcctggagctctgagaacagaaggagagtcattcgtgttgagtttccacaaatactgtcttgagttttgcaataaaagtgggatagcagagttgagtgagccataggctgagttctctcttttgtctcctaagtttttatgactacaaaaatcagtagtatgtcctgaaataatcattaaactgtttgaaagtatgactgcttgccatgtagataccatggcttgctgaataatcagaagaggtgtgactcttattctaaaatttgtcacaaaatgtcaaaatgagagactctgtaggaacgagtccttgacagacagctcaaggggtttttttcctttgtctcatttctacatgaaagtaaatttgaaatgatcttttttattataatagtagaaatacagttgggtttgaactatatgttttaatggccacggttttgtaagacatttggccctttgttttcccagttattactcgcttgtaattttatatcgccagcaatggactgaaacggtccgcaacctcttctttacaactgggtgacctcgcggctgtgccagccatttggcgttcaccttgccgctaagggccgtgtgaacccccgaggtagcatcccttgctccgcgtggaccactttcctgaggcacagtgataggaacagagccactaatctgaagagaacagagatgtgacagactacactaatgttagaaaaacaaggaaagggtgacttattggagatttcagaaataaaatgcatttattattatattcccttattttaattttctattagggaattagaaagggcataaactgctttatccagtgttatattaaaagctttttttttttcagtgcta

SEQ ID NO: 11 - DNA, 19 bp, Artificial
  oligonucleotide primer that amplifies mouse anti-human monoclonal antibody cDNA
gaggttccttgaccccagt

SEQ ID NO: 12 - DNA, 19 bp, Artificial
  oligonucleotide primer that amplifies mouse anti-human monoclonal antibody cDNA
cgattcccagttcctcaca

SEQ ID NO: 13 - DNA, 20 bp, Artificial
  oligonucleotide primer that amplifies mouse anti-human monoclonal antibody cDNA
aacgtccacggatagttgct

SEQ ID NO: 14 - DNA, 19 bp, Artificial
  oligonucleotide primer that amplifies mouse anti-human monoclonal antibody cDNA
cagaacagcatgggcttca

SEQ ID NO: 15 - protein, 214 aa, Artificial
  mouse anti-human monoclonal antibody
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, Artificial
  mouse anti-human monoclonal antibody
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, Artificial; identical to SEQ ID NO: 15
  mouse anti-human monoclonal antibody

SEQ ID NO: 18 - protein, 449 aa, Artificial; identical to SEQ ID NO: 16
  mouse anti-human monoclonal antibody

SEQ ID NO: 19 - protein, 5 aa, Artificial
  mouse anti-human monoclonal antibody
EEQYN

SEQ ID NO: 20 - protein, 5 aa, Artificial
  mouse anti-human monoclonal antibody
VSITC

SEQ ID NO: 21 - protein, 5 aa, Artificial
  mouse anti-human monoclonal antibody
LIVSL

SEQ ID NO: 22 - protein, 10 aa, Artificial
  mouse anti-human monoclonal antibody
SGTASVVCLL

SEQ ID NO: 23 - protein, 5 aa, Artificial
  mouse anti-human monoclonal antibody
TKPRE

(19) United States
(12) Patent Application Publication
(10) Pub. No.: US 2004/0219633 A1
(43) Pub. Date: Nov. 4, 2004

(54) Method of producing recombinant antibodies

(75) Inventors: Reinier Lh Bolhuis, Leimuiden (NL); Thorsten Wohl, Planegg (DE); Volker Bottger, Germering (DE)

Correspondence Address: ROTHWELL, FIGG, ERNST & MANBECK, P.C., 1425 K STREET, N.W., SUITE 800, WASHINGTON, DC, 20005, US

(73) Assignee: WILEX AG, Muenchen (DE)

(21) Appl. No.: 10/635,908

(22) Filed: Aug. 7, 2003

Related U.S. Application Data
(63) Continuation-in-part of application No. PCT/EP02/01283, filed on Feb. 7, 2002.
(60) Provisional application No. 60/327,008, filed on Oct. 5, 2001.
(60) Provisional application No. 60/266,853, filed on Feb. 7, 2001.

(51) Int. Cl. A61K 39/395; C07H 21/04; C07K 16/28; C12N 5/06

(52) U.S. Cl. 435/69.1; 435/320.1; 435/334; 530/388.22; 536/23.53

(57) ABSTRACT

The invention relates to novel nucleic acid sequences which encode an antibody suitable in the field of tumor diagnostics and therapeutics. Further, a method of producing recombinant antibodies is provided, wherein the novel nucleic acid sequencs are employed.

The invention relates to novel nucleic acid sequences which encode an antibody suitable in the field of tumor diagnostics and therapeutics. Further, a method of producing recombinant antibodies is provided, wherein the novel nucleic acid sequencs are employed.

The monoclonal antibody G250, subclass IgG1, recognizes an antigen preferentially expressed on membranes of renal cell carcinoma cells (RCC) and not expressed in normal proximal tubular epithelium. The antibody G250 was obtained by immunizing a mouse with cell homogenates from primary RCC lesions obtained from different patients (Oosterwijk et al., Int. J. Cancer 38 (1986), 489-494).

The antibody G250 as well as chimeric derivatives has been used in clinical studies (Steffens et al., J. Clin. Oncol. 15 (1997), 1529-1537). The nucleic acid sequence coding for the antigen-binding site of G250, however, has not been published yet.

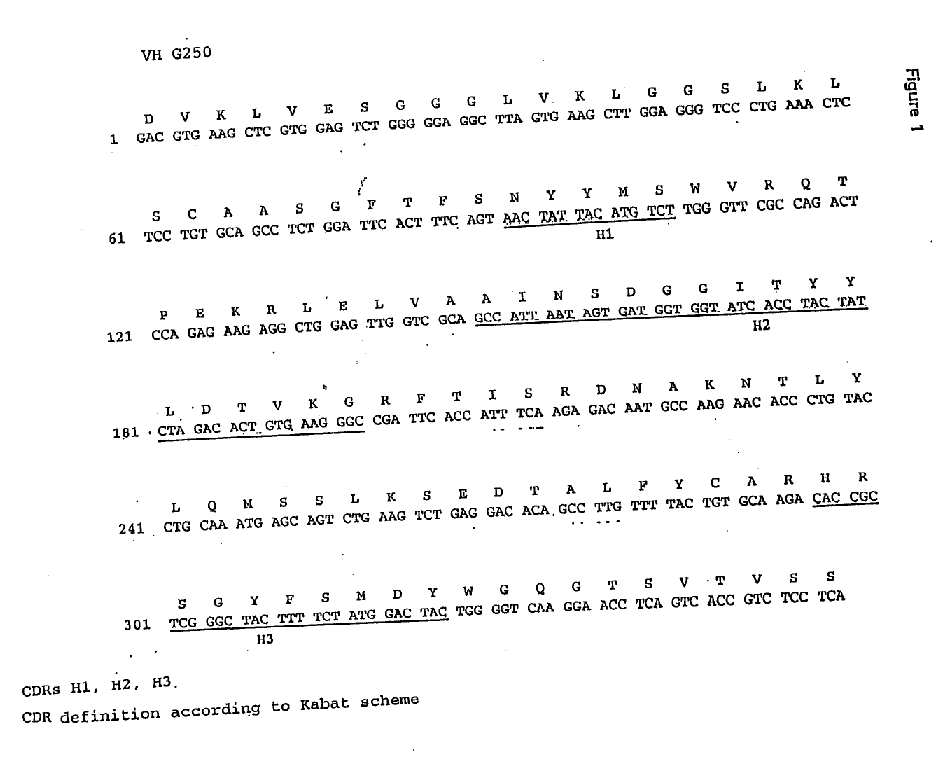

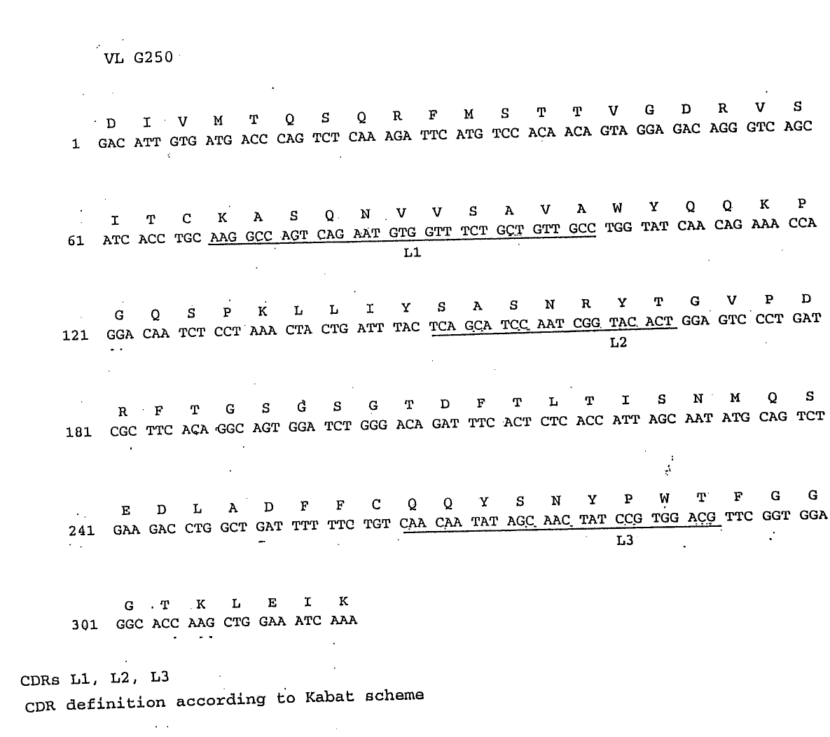

Thus, a subject matter of the present invention is a nucleic acid encoding the antigen-binding site of the heavy chain of an antibody comprising a nucleotide sequence encoding the CDR3 region as shown in FIG. 1 (designated H3).

The nucleic acid sequence furthermore preferably comprises a nucleotide sequence encoding the CDR2 region as shown in FIG. 1 (designated H2) and/or a nucleotide sequence encoding the CDR1 region as shown in FIG. 1 (designated H1). More preferably, the nucleotide sequences encoding the CDR3, CDR2 and CDR1 regions are arranged in a manner wherein a polypeptide encoded by the nucleotide sequence is capable of forming an antigen-binding site having substantially the same characteristics as the heavy chain antigen-binding site of the monoclonal antibody G250.

A further aspect of the present invention relates to a nucleic acid encoding the antigen binding site of the light chain of an antibody comprising a nucleotide sequence encoding the CDR3 region as shown in FIG. 1 (designated L3).

Preferably the nucleic acid further comprises a nucleotide sequence encoding the CDR2 region as shown in FIG. 1 (designated L2) and/or a nucleotide sequence encoding the CDR1 region as shown FIG. 1 (designated L1).

More preferably, the nucleic acids encoding the CDR3, CDR2 and CDR1 region are arranged such that a polypeptide encoded by the nucleic acid has substantially the same antigen-binding characteristics as the light chain antigen binding site of the antibody G250.

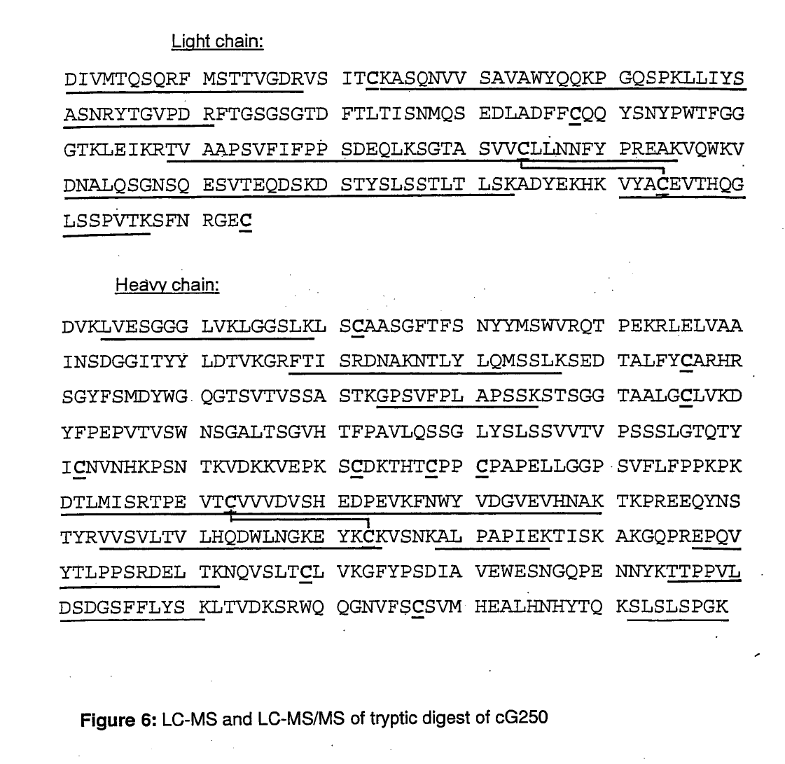

In the nucleic acid of the invention the complement determining regions CDR3, CDR2 and CDR1 are preferably separated by nucleotide sequence portions encoding so-called framework regions of antibodies. The framework regions may be derived from any species, e.g. from mouse (as shown in FIG. 1 or FIG. 6), it is, however, possible to use framework regions from different species, e.g. human framework regions. It should be noted that also the CDR1, CDR2 and/or CDR3 regions may be modified, e.g. by modifying the nucleotide sequence resulting in a modified nucleotide sequence encoding a polypeptide sequence differing from the polypeptide sequence as depicted in FIG. 1 or FIG. 6, provided that the antigen-binding specificity remains substantially the same. More preferably, however, the nucleic acid sequences of the heavy chain and light chain CDR3 sequence and of the CDR2 and CDR1 sequence, if present, have the nucleotide sequence as depicted in FIG. 1 or/and the nucleic acid sequences have the nucleotide sequence as depicted in FIG. 1.

Further, the light chain or/and the heavy chain may have the amino acid sequence as depicted in FIG. 6. Thus, the nucleic acid of the present invention may comprise a sequence encoding the light chain or/and the heavy chain as shown in FIG. 6.

The nucleic acid sequences of the present invention may be located on a recombinant vector comprising at least a copy of a heavy chain nucleic acid and/or at least a copy of a light chain nucleic acid. The heavy chain nucleic acid and the light chain nucleic acid are preferably in operative linkage with an appropriate expression control sequence, particularly an expression control sequence which is functionally in eukaryotic cells. The heavy chain and the light chain nucleic acid may be located on the same vector in operative linkage with a single expression control sequence or with separate expression control sequences which may be the same or different. Alternatively, the heavy chain nucleic acid sequence and the light chain nucleic acid sequence may be located on different recombinant vectors, each in operative linkage with a separate expression control sequence.

Thus, a further aspect of the present invention is a recombinant vector system comprising at least one copy of a nucleic acid encoding the antigen-binding site of the heavy chain of an antibody comprising a nucleotide sequence encoding the CDR3 region (designated H3), or/and encoding the CDR2 region (designated H2), or/and-encoding the CDR1 region (designated H1), as shown in FIG. 1 or/and FIG. 6, and at least one copy of a nucleic acid encoding the antigen-binding site of the light chain of an antibody comprising a nucleotide sequence encoding the CDR3 region (designated L3), or/and encoding the CDR2 region (designated L2), or/and encoding the CDR1 region (designated L1), as shown in FIG. 1 or/and FIG. 6, wherein the nucleic acid encoding the antigen-binding site of the heavy chain and of the light chain have separate expression control sequences.

The recombinant vector system comprises a first recombinant vector comprising at least one copy of a nucleic acid encoding the antigen-binding site of the heavy chain and a second recombinant vector comprising at least one copy of a nucleic acid encoding the antigen-binding site of the light chain.

Alternatively, in the recombinant vector system, at least one copy of the nucleic acid encoding the antigen-binding site of the heavy chain and of the light chain are located on the same recombinant vector.

Further, the present invention comprises a method for the recombinant production of a polypeptide having an antigen-binding site comprising:

(a) providing a nucleic acid as defined above or/and providing a recombinant vector system as defined above,

(b) introducing the nucleic acid into a suitable host cell,

(c) culturing the host cell under suitable conditions in a medium whereby an expression of the nucleic acid takes place and

(d) obtaining the expressed product from the medium and/or the host cell.

Preferably, the host cell is a eukaryotic cell, particularly a mammalian cell. For example, the host cell may be a non-producer hybridoma cell or a CHO cell.

Between steps (a) and (b) of the method as outlined above a modification of the nucleic acid sequence may take place, wherein the modification substantially does not alter the amino acid sequence of the antigen-binding site of the polypeptide to be expressed. The expressed product obtained by the method as outlined above may be used for the preparation of a diagnostic or therapeutic agent. Thereby it is possible to couple the antigen-binding polypeptide to a diagnostic marker, e.g. a marker which is useful for in vitro diagnostic methods using a sample obtained from a patient, e.g. a body fluid or a tissue section, or for quality control. Further, the expressed product may be coupled to a diagnostic marker which is suitable for in vivo applications, e.g. a radioactive marker which is suitable for radioimaging procedures. For therapeutical applications the expressed product may be coupled to a cytotoxic agent, e.g. a radionuclide, a toxin such as cholera toxin or ricin.

The expressed product which is obtained by the method as outlined above is a polypeptide having an antigen-binding site. For example, the expressed product may be selected from antibodies, e.g. chimerized antibodies, humanized antibodies, heterobispecific antibodies, single chain antibodies etc. and from antibody fragments, e.g. antibody fragments containing an antigen-binding site wherein said antibody fragments may be obtained by proteolytic digestion of whole antibodies or by recombinant techniques.

The manufacture of chimeric antibodies is described e.g. by Morrison et al. (Proc. Natl. Acad. Sci. USA 81 (1984), 6851-6855), which is herein incorporated by reference. The manufacture of humanized antibodies is described, e.g. in Jones et al. (Nature 321 (1986), 522-525), Riechmann et al. (Nature 332 (1988), 323-329) and Presta (Curr. Opin. Struct. Biol. 2 (1992), 332-339) which are herein incorporated by reference.

Single chain antibodies or antibody fragments may be prepared as described in Hoogenboom et al. (Immunol. Rev. 130 (1992), 41-68), Barbas III (Methods: Companion Methods Enzymol. 2 (1991), 119) and Plückthun (Immunochemistry (1994), Marcel Dekker Inc. Chapter 9, 210-235), which are herein incorporated by reference.

Further, the present invention is explained in detail by the following examples:

### EXAMPLE 1

Isolation, cloning and sequencing of the G250 tumor-associated antigen-specific immunoglobulin variable heavy and light chain domains from the G250 monoclonal antibody producing hybridoma.

General Strategy

The variable region genes for the heavy and light chains, which determine the binding specifity of the antibody, were cloned from the G250 murine hybridoma using standard cloning techniques as decribed in Molecular Cloning; A Laboratory Manual (Cold Spring Harbour Press, Cold Spring Harbour, N.Y.) by Maniatis, T. et al.

The strategy for cloning the variable regions for the heavy and light chain genes from the G250 hybridoma was achieved by PCR amplification of cDNA obtained from the G250 monoclonal antibody producing hybridoma cells.

Cloning of G250 VH and VL cDNA

Obtaining the G250 VH and VL chain sequences from the G250 monoclonal antibody producing hybridoma was achieved by PCR (Maniatis, T. et al.) amplification of cDNA obtained from the respective clone.

To obtain cDNA, total RNA was isolated from the G250 producing hybridoma cells according to the method by Chomczynski et al. (Chomczynski, P. and Sacchi, N., Anal. Biochem. 162 (1987), 156-159) and converted into cDNA essentially as described by Maniatis et al.

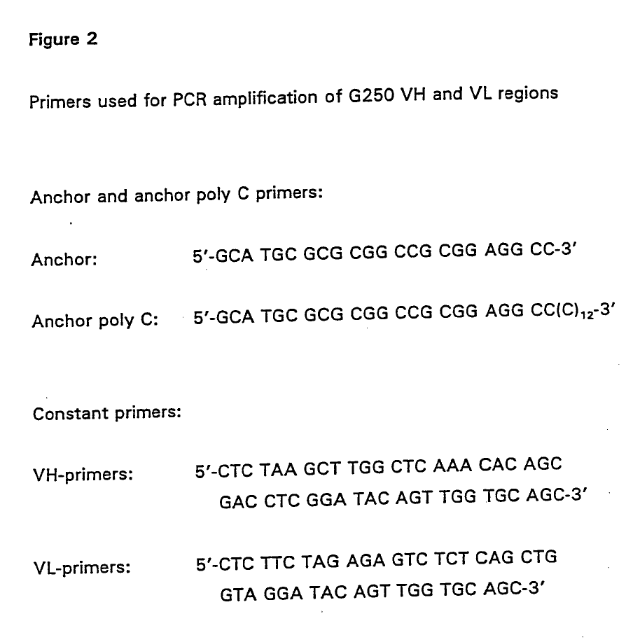

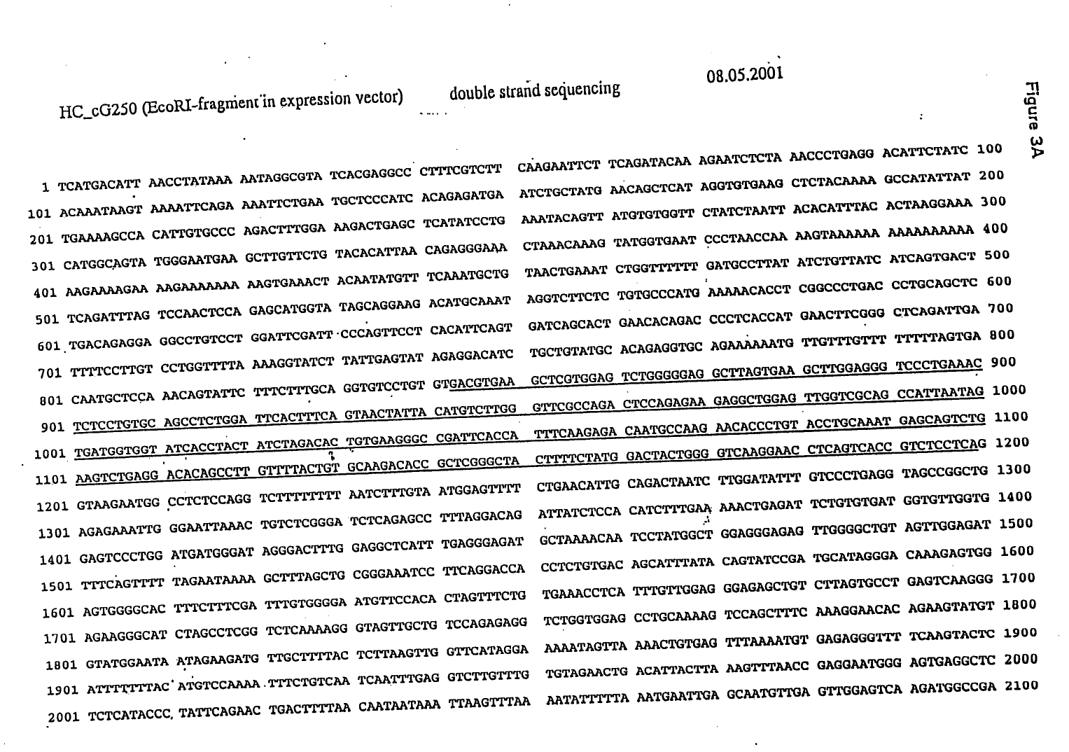

Amplification of cDNA sequences by PCR is possible only, if the sequence of the gene of interest is known. In general, for PCR two primers complementary to the 5′-end and the 3′-end of the sequence are used as the initiation point of DNA synthesis. Because the sequence of the 5′-ends of the VH and VL chain from the G250 monoclonal antibody producing hybridoma cells were unknown, the PCR method, referred to as RACE (rapid amplification of cDNA ends) was used to amplify the VH and VL chain. This was achieved by employing anchor and anchor-poly-C primers and the constant VH and VL-primers as shown in FIG. 2. The VH and VL fragments were purified and ligated into pGEM 11 as described by Maniatis et al. A ligation mixture was introduced into bacteria, which were selected and expanded. DNA was isolated from the selected bacterial colonies and analyzed by restriction enzyme digestion to confirm the presence of the amplified VH and VL fragments. Three positive colonies were subjected to DNA sequencing. The sequences of these three individual clones were compared and found to be identical.

Portions of the resulting sequences including the antigen-specific CDR regions are shown in FIG. 1.

### EXAMPLE 2

Sequencing of cDNA sequences encoding variable heavy and light chain domains of the G250 monoclonal antibody

Strategy

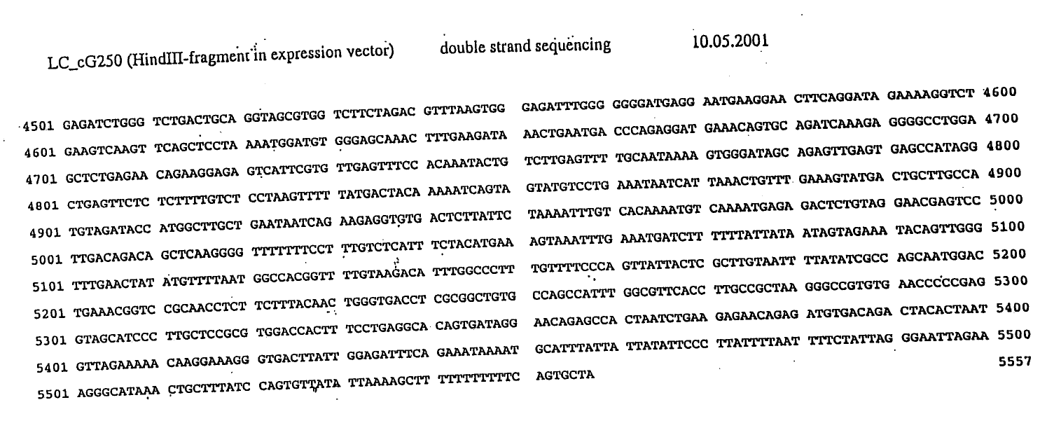

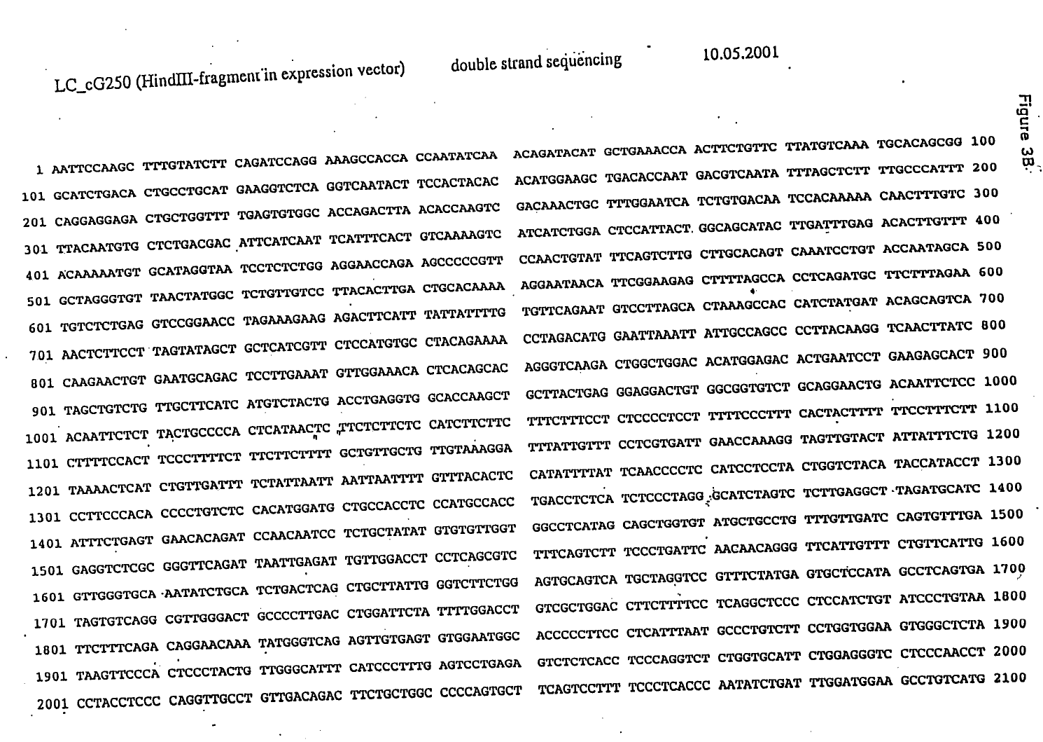

The G250 VH and VL chain cDNA sequences were obtained as described in co-pending U.S. patent application 60/327,008, example 3. The resulting cDNA fragments, a 2.3 kb EcoRI heavy chain variable region fragment and a 5.5 kb HindIII light chain variable region fragment were cloned into suitable expression vectors which contain the human G1 constant region (for the H-chain) or the human Kappa constant region (for the L-chain), respectively, and genes conferring resistance to selectable markers. Competent bacteria (E.coli TG1) were transformed with the plasmids. Ampicillin resistant clones were selected and expanded. Plasmid DNA was isolated using the Nucleobond AX 500 Maxiprep Kit from Machery & Nagel (Germany). The isolated DNA was subjected to cycle sequencing using the DYEnamic ET Terminator Cycle Sequencing Kit (Amersham Biosciences, Freiburg, Germany) and the resulting DNA molecules labeled with multiple fluorescent dyes were analyzed using the ABI PRISM Model 377 DNA Sequencer (Applied Biosystems, Weiterstadt, Germany). The employed sequencing primers are shown in the following. For sequencing of the full length inserts, the 2.3 kb EcoRI and 5.5 kb HindIII, respectively, a primer-walking approach was applied. The obtained Sequences of both the CDR's as well as the heavy and light chain is shown in FIG. 3.

Primers used for Cycle Sequencing of the variable region of G250 heavy (H) and light (L) chain

| | |
|---|---|
| RightH | GAG GTT CCT TGA CCC CAG T |
| LeftH | CGA TTC CCA GTT CCT CAC A |
| RightL | AAC GTC CAC GGA TAG TTG CT |
| LeftL | CAG AAC AGC ATG GGC TTC A |

The sequencing results are shown in FIG. 3. The primer sequences are underlined. The CDR sequences are boxed.

### EXAMPLE 3

Polypeptide binding specifity and peptide mass fingerprinting

Binding specifity of the gene product encoded by the sequences identified in examples 1 and 2 was tested by means of a sandwich-type ELISA using a G250 anti-idiotypic mouse monoclonal antibody as capture and detection antibody and chimeric G250 antigen for the calibration curve. ELISA analysis demonstrated the presence of G250 antibodies (>6 μg/ml) in the supernatant of a transfected cell line.

For protein chemical analysis supernatant from a cell culture expressing G250 antibody was collected. The IgG fraction was enriched using a one-step protein G-chromatography. An aliquot of the eluted fraction was subjected to SDS-PAGE. A total of five bands with the apparent molecular weight of heavy or light chains were subjected to a peptide mass fingerprint analysis by MALDI mass spectroscopy. An analysis of heavy and light chain peptides demonstrated identity of the antibody produced by the cell line with the original mouse G250 antibody. The analysis confirmed the presence of peptide fragments specific for CDR1, CDR2, and CDR3 of the heavy chain and CDR2 of the light chain.

### EXAMPLE 4

Sequence Verification and Mass Spectrometric Characterization of the Recombinant Antibody WX-G250

Introduction

WX-G250 represents a chimeric antibody (cG250) directed against the antigen G250 (carbonic anhydrase 9), a protein expressed on kidney tumor (RCC) cells. G250 is a potential target protein for kidney tumor. The antibody cG250 consists of two identical heavy chains of approx. 50 KD and two identical light chains of approx. 25 KD.

This study was carried out to examine three different aspects of WX-G250 structure:

1) Verification of the amino acid sequence

2) Characterization of the configuration of its disulfide bridges

3) Examining posttranslational modifications like glycosylations

Abbreviations

KD kilo Dalton

MALDI Matrix-assisted laser desorption ionization

MS Mass spectrometry

TOF Time of flight

PMF Peptide mass fingerprint

SDS Sodium dodecylsulfate

PAGE Polyacrylamide gel electrophoresis

MW Molecular weight

DTT Dithiothreitol

MH+ single charged protonated peptide mass

exp experimental

th theoretical

RP Reversed Phase

HPLC High Performance Liquid Chromatography

hc Heavy Chain

Ic Light Chain

Materials and Methods:

1. Materials

1.1. Desalting: C18 ZipTip, C4 ZipTip, Millipore, Bedford, Mass., USA.

1.2. MALDI mass spectrometry: Voyager STR, Applied Biosystems, Foster City, Calif., USA

1.3. Nano-ESI mass spectrometry: QSTAR Hybrid Quadrupole-TOF LC/MS/MS (022222-44) Perkin-Elmer Sciex Instruments, Foster City, Calif., USA

1.4. HPLC of enzymatic digests: HP 1100 (Agilent)

1.5. HPLC of reduced and alkylated antibody: HP 1090 (Agilent)

1.6. LC-MS/MS: Ultimate nano-LC system (Dionex), EsquireLC ion trap mass spectrometer (Bruker Daltonics)

1.7. Proteases: Trypsin, Lys-C, Glu-C, Asp-N, Roche Diagnostics, Basel, Switzerland

1.8. N-Glycosidase F: Roche Diagnostics, Basel, Switzerland

1.9. SDS-PAGE: 12% Polyacrylamide minigel (BioRad), Gel chamber (BioRad), Laemmli buffer, Coomassie Blue G250 (Sigma), MW-Marker (Invitrogen)

1.10. All used chemicals were from Sigma Aldrich, Munich, Germany

2. Methods

Mass Spectrometric Analysis of Intact WX-G250 (MALDI-MS Analysis)

The sample was desalted using a C4-ZipTip (Millipore according to the manufacturers protocol. The eluted sample was mixed 1:1 with the matrix solution sinapinic acid and analyzed by MALDI-MS. The mass spectrometer (Voyager STR, Applied Biosystems) was calibrated externally with an IgG standard (Sequazyme™ Peptide Mass Standard Kits, Applied Biosystems).

Mass Spectrometric Analysis of Light and Heavy Chains after Reduction and Alkylation of Intact WX-G250 (MALDI-MS, ESI-MS)

The sample was reduced with dithiothreitol (DTT, 7 μg/μl) for 20 minutes at 55° C. and alkylated with iodacetamide (IAA, 18,6 μg/μl) for 20 minutes at room temperature. The sample was desalted by a C4-ZipTip (Millipore) according to the manufacturers protocol. The eluted sample was mixed 1:1 with matrix solution sinapinic acid and analyzed by MALDI-MS. The mass spectrometer (Voyager STR, Applied Biosystems) was calibrated externally with a protein standard mixture (Sequazyme™ Peptide Mass Standard Kits, Applied Biosystems).

For ESI-MS measurements a QStar (Sciex-Applied Biosystems) equipped with a nanospray source (Protana) was used. The QStar is a QTOF instrument using a TOF as ion mass discriminator. The desalted and lyophilized sample was dissolved in 50% acetonitrile, 0.5% formic acid (v/v). 2 μl of the sample was centrifuged into a nanospray needle (Protana). The needle was then built in into the nanospray source and connected with an electrode. The needle was broken very carefully to allow a homogenous ion spray. The voltage was increased until a continuous nanospray was reached.

Light and Heavy chains of 10 μg of the reduced and alkylated WX-G250 antibody were separated by reversed phase HPLC. A HP1090 HPLC, and a RP C4 column (Vydac Protein C4, 2 mm×250 mm) was used for this separation. The gradient conditions were the following: solvent A: 0.1% TFA in water, solvent B: 0.1% TFA in 80% acetonitrile; gradient: 0 min. 30% B, 5 min. 30% B, 60 min. 75% B, 75 min. 95% B, 85 min. 95% B, 90 min. 30% B. The flow was 1 ml/min.

The fractions were subsequently analyzed by SDS-PAGE.

Mass Spectrometric Analysis of Proteolytic Digests of WX-G250 with Trypsin, LysC, AspN and GluC by MALDI Peptide Mass Fingerprinting (PMF)

The endoprotease trypsin cleaves specifically C-terminal of the basic amino acid residues lysine (K) and arginine (R). The endoprotease LysC cleaves specifically C-terminal of the amino acid residue lysine (k). AspN cleaves specifically N-terminal of the amino acid residue aspartic acid (D). GluC cleaves specifically C-terminal of the amino acid residue glutamic acid (E). Cyanogen bromide (BrCN) cleaves specifically C-terminal of the amino acid residue methionine. For cyanogen bromide cleavage of the intact antibody WX-G250 was incubated overnight at room temperature in 70% formic acid added with 100 mM BrCN.

The resulting peptide mixtures after digest represent characteristic fingerprints for each protein, depending on the corresponding protein sequence.

For the digest with the enzymes trypsin, LysC, and AspN the same digestion protocol was used: WX-G250 was digested over night at 37° C. (c: 1 μg/μl in 2 M urea, 400 mM NH4HCO3) after reduction (45 mM Dithiothreol (DTT) in 8 M urea, 400 mM NH4HCO3, 30 min, 50° C.) and alkylation (100 mM lodacetamid in 8 M urea, 400 mM NH4HCO3, 30 min 50° C. at room temperature). For GluC the only difference to the protocol described above was that the incubation was done at room temperature. The samples were desalted with a C18-ZipTip (Millipore) according to the manufacturers protocol. The eluted peptides were mixed 1:1 with matrix solution (2,5-dihydroxybenzoic acid (DHB): 2-hydroxy-5-methoxybenzoic acid 9:1) and analyzed by MALDI-MS. The resulting peptide masses were compared with the respective tryptic in-silico digests using the MS digest program of Protein Prospector V3.2.1. For the in-silico digest two miscleavages (lysine or arginine where trypsin has not cleaved) were allowed. For the MALDI-TOF-MS measurements a Voyager STR (Applied Biosystems) was used. The used mass range of the MALDI-TOF-MS analysis was from 700-4200 Da. The autotryptic masses of 805.41 Da and 2163.05 Da were used for internal calibration. After internal calibration the mass accuracy was better than 50 ppm.

MALDI PMF without Reduction after Cleavage of WX-cG250 with Trypsin, LysC, AspN, GluC, and BrCN

For the determination of disulfide bridges the complete cG250 (Heavy and Light chain) was digested without prior reduction and alkylation.

Detection of other Posttranslational Modifications by MALDI MS of HPLC-separated Peptides (Trypsin, AspN, LysC, and GluC Digests)

Due to suppression effects in MALDI MS especially glycopeptides often are not detectable in a complex mixture. Due to the low mass difference of 1D and the overlapping isotope patterns of peptides differing in 1D, deamidated peptides cannot be detected in presence of non-deamidated peptides. Therefore, the peptides were separated by HPLC.

Digestion was performed according to the manufacturers protocol. WX-G250 (c: 0.5 μg/μl) was denatured in 1% SDS, 100 mM PBS, pH 7.3, 1% mercaptoethanol and diluted in 0.1% SDS, 1% CHAPS, 100 mM PBS, pH 7.3, 1% mercaptoethanol (c: 0.05 μg/μl) WX-G250 was incubated with five units of N-Glycosidase F overnight. The solution was delivered to Wilex for isoelectric focussing.

Peptide mixtures were separated on a 300 μm×150 mm capillary HPLC column using a linear acetonitrile gradient with a slope of 0.57% B/min starting from 2% B to 45% B in 75 minutes. Solvent A: was 5% acetonitrile, 0.1% trifluoracetic acid, solvent B 80% acetonitrile, 0.1% trifluoracetic acid, the column was from LC-Packings, filled with Vydac RP18, 5 μ, 300 Å material. The HPLC system used was a HP 1100 system from Agilent.

LC-MS and LC-MS/MS of Tryptic Digest of cG250

The tryptic peptide mixture of cG250 was separated using a 75 μm×150 mm capillary HPLC column (RP18, Dionex) at a flow rate of 200 nl. MS was performed with a quadrupole ion trap (Esquire, Bruker Daltonics). The two most intensive signals of each spectrum were fragmented (MS/MS).

SDS-PAGE

To evaluate the efficiency of the separation of the Light and Heavy chains by RP-HPLC the HPLC fractions were applied to a 12% SDS-PAGE gel. 50% of the HPLC fractions were dried in a vacuum concentrator and subsequently solved in SDS-PAGE sample loading buffer (25 mM Tris/HCI, pH 7.5, 2% SDS, 1% DTT, 15% glycerole). As MW standard a protein standard from Invitrogen was used (10-220 KD). The gel was run at 150 V for 1.5 hours according to Laemmli et al. Then it was stained with Coomassie Blue G250 for 1 hour.

Edman Sequencing of Selected HPLC Fractions after Enzymatic Digest

To verify the posttranslational modifications of the trypsin digest and fractions of the LysC digest were analyzed by automated Edman sequencing.

Results:

Mass Spectrometric Analysis of Intact WX-G250 (MALDI-MS Analysis)

The linear mode MALDI-MS spectrum showed signals of the single to triple charged ions of the intact antibody (MWexp.: 149135 D, MWth.: 147424 D). The mass spectrometer was externally calibrated with an antibody standard (Applied Biosystems). The difference between the theoretical and the experimentally determined MW might result from glycosylation. The mass accuracy in this MW range is approximately 100-150 ppm.

Mass Spectrometric Analysis of Light and Heavy Chains after Reduction and Alkylation of Intact WX-G250 (MALDI-MS, ESI-MS)

To detect the masses of the separated Light and Heavy chains of WX-G250 the antibody was reduced and alkylated as described in Materials and Methods, desalted by ZipTip (Millipore), and then applied to the mass spectrometer.

The MALDI-MS spectrum shows signals of the single and double charged ions of the Light chain. (MWexp.: 23886 D, MWth.: 23873 D) and a signal of a protein (Heavy chain) at m/z: 51507 D (MWth,: 49839 D). The MW of the Light chain represents the theoretically expected mass (difference: 13 D), whereas between the theoretical and experimental MW of the Heavy chain a significant difference of 1.668 D was observed. This finding leads to the assumption that the antibody is glycosylated only at its Heavy chain.

Due to an improved resolution compared to MALDI-MS three different isoforms of WX-G250 were detected. The ESI-MS spectrum showed strong signals of the 15-fold to 34-fold charged ions of the light chain and very weak signals of the 53-fold to 57-fold charged ions of three different proteins at about 52 KD (light chain: 23869 D, other proteins: 51036 D, 51201 D, 51328 D; theoretical masses: Ic: 23873, hc: 49839). The mass difference between the three Heavy chain isoforms found was 165 D and 128 D. The latter mass difference corresponds to a lysine residue (128 D). The accuracy of the ESI-MS was better than 50 ppm.

10 μg of the reduced and alkylated antibody was separated by reversed phase HPLC. The fractions were analyzed by SDS PAGE and MALDI-TOF MS. Fractions 4 and 5 contained light and heavy chain of WX-G250.

Mass Spectrometric Analysis of Proteolytic Digests of WX-G250 with Trypsin, LysC, AspN and GluC by MALDI Peptide Mass Fingerprinting (PMF)

The digests of the whole antibody were performed to confirm the amino acid sequence.

Peptides derived from Light and Heavy chains of WX-G250 from the PMFs digested with different enzymes were found.

Some of the peptides of the tryptic and LysC digests containes lysine or arginine residues. These miscleavages are not unusual and occur probably due to lack of enzyme access at certain cleavage positions.

To check if the C-terminal part of the heavy chain was somehow modified, the whole antibody was incubated with BrCN in 70% formic acid overnight at room temperature. For this digest cG250 was not reduced and alkylated. One peptide derived from the Light chain was found as well. Some other peptides (MH+: 816.41 D, 871.40 D, 877.40 D, 887.41 D, 1277.65 D, 1305.60 D, 1333.64 D) were found as well. However, these peptides could not be explained by the theoretical digest of WX-G250.

Probably these peptides were due to chemical modifications as obtained by the homoserine lactone from the Light chain.

MALDI PMF without Reduction after Cleavage of WX-cG250 with Trypsin, LysC, AspN, GluC, and BrCN

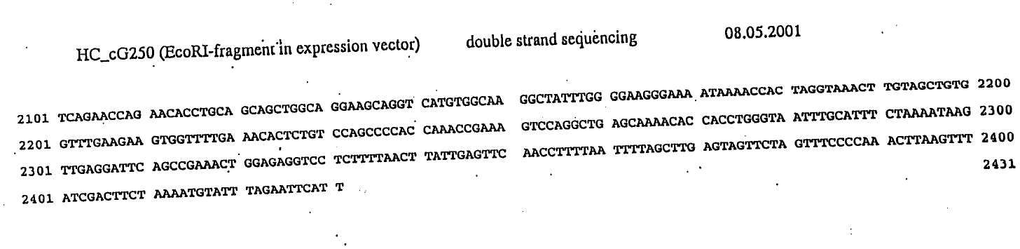

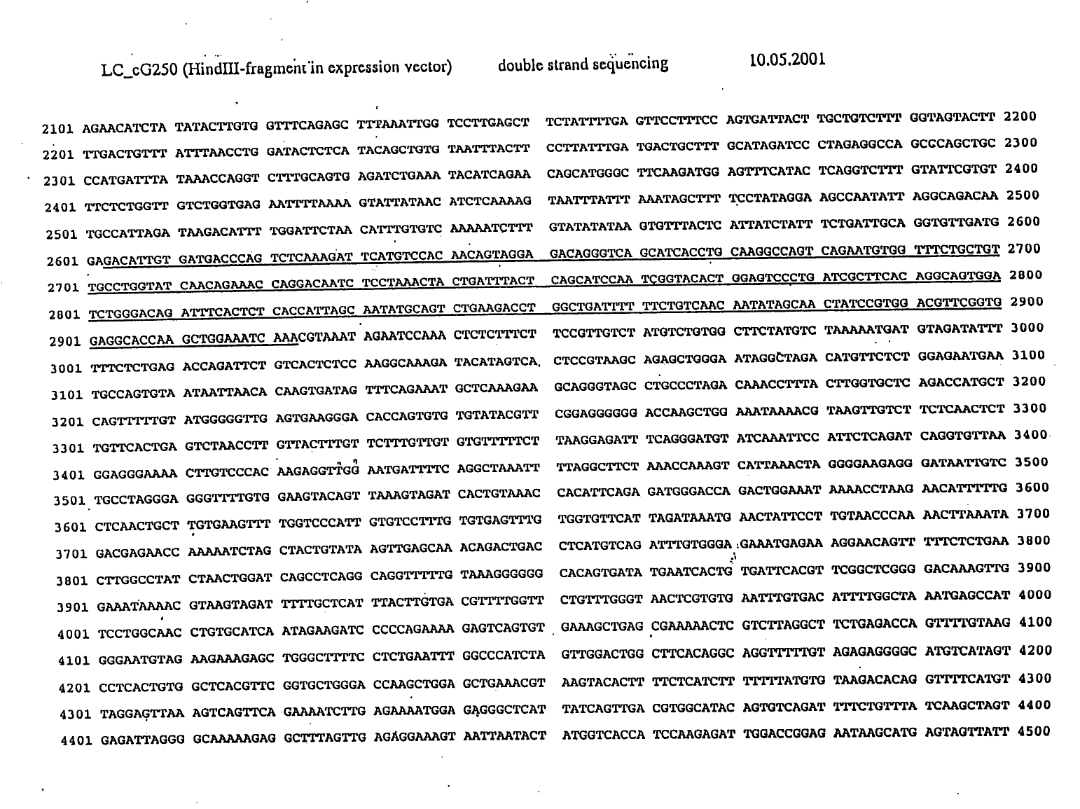

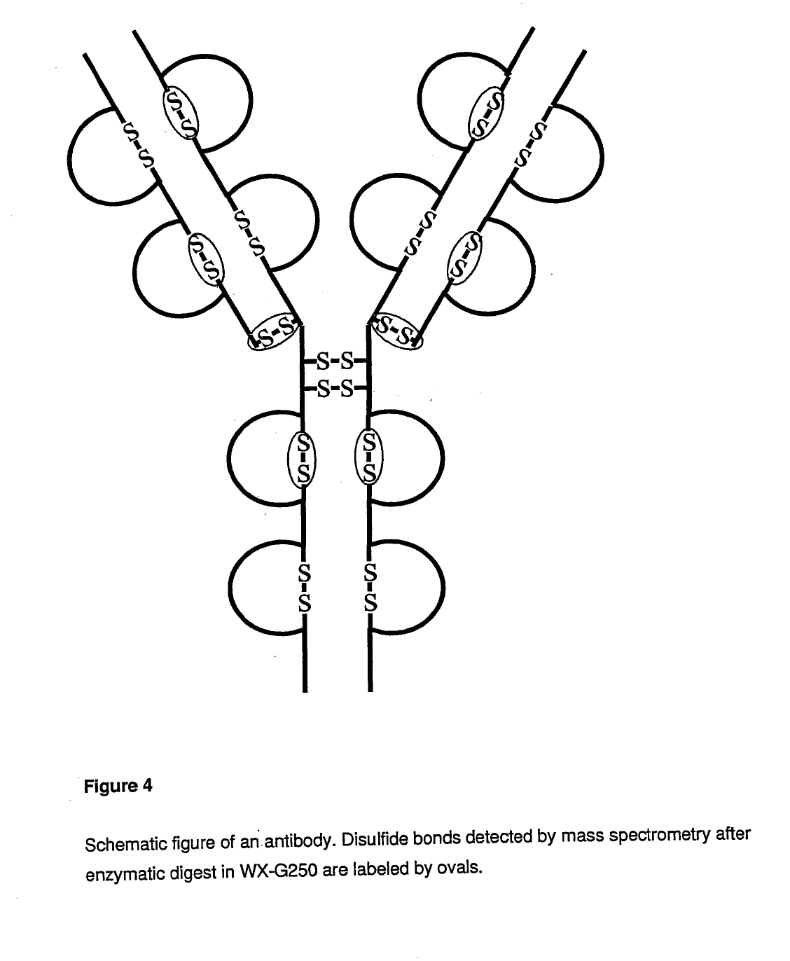

The disulfide bridges found in the complete cG250 (Heavy and Light chain) are summarized in Table 1. A schematic summary is given in FIG. 4.

The mass difference between the carbamidomethylated cysteins (see reduced and alkylated peptides) and the unmodified cysteine residues is 57 D.

Table 1: Disulfide Bridges in WX-cG250

Light Chain:

| | | | | |
|---|---|---|---|---|
| 3559.1 | 127-142/191-207 | Cys134-Cys194 | linear mode | tryptic |
| | | | | digest |
| 3824.4 | 127-142/189-207 | Cys134-Cys194 | linear mode | tryptic |
| | | | | digest |
| 5256.8 | 19-24/62-103 | Cys23-Cys88 | linear mode | tryptic |
| | | | | digest |
| 6046.6 | 19-24/55-103 | Cys23-Cys88 | linear mode | tryptic |
| | | | | digest |
| 6251.9 | 10-24/62-103 | Cys23-Cys88 | linear mode | tryptic |
| | | | | digest |
| 3559.0 | 127-142/191-207 | Cys134-Cys194 | reflector mode | tryptic |
| | | | | digest |
| 3824.4 | 127-142/189-207 | Cys134-Cys194 | reflector mode | tryptic |
| | | | | digest |
| 3887.4 | 127-145/191-207 | Cys134-Cys194 | reflector mode | LysC |
| | | | | digest |
| 4152.7 | 127-145/189-207 | Cys134-Cys194 | reflector mode | LysC |
| | | | | digest |

Heavy Chain:

| | | | | |
|---|---|---|---|---|
| 3464.1 | 20-38/88-98 | Cys22-Cys96 | linear mode | tryptic |
| | | | | digest |
| 4744.4 | 20-38/77-98 | Cys22-Cys96 | linear mode | tryptic |
| | | | | digest |
| 2311.1 | 258-276/323-324 | Cys263-Cys323 | LC-MS/MS | tryptic |
| | | | | digest |

Intermolecular disulfide bridge:

| | | | | |
|---|---|---|---|---|
| 1842.8 | 208-214 (Ic)/ | Cys214-Cys222 | reflector mode | LysC |
| | 216-224 (hc) | | | digest |

Detection of other Posttranslational Modifications by MALDI MS of HPLC-separated Peptides (Trypsin, AspN, LysC, and GluC Digests)

One N-glycosylation site was characterized at N 299 (hc). The sequence showed the NST motif, which represents a potential N-glycosylation site. According to the data required by MALDI-MS mass three different variants of complex type N-glycosylation (4×GlcNAc, 3-5 Hexose, 1×Fucose) were found. The three isoforms differed by one and two hexoses, respectively (mass difference: 162 D).

Two deamidation sites were located. Asparagine was deamidated to aspartic acid. One of these sites was located in the Light chain (amino acid residues N 137 or N 138). Due to the two neighbored asparagine residues it could not be detected which of them was deamidated.

Another deamidation site was found in Heavy chain, position N 317. It was detected by MALDI-MS in a tryptic peptide after HPLC fractionation.

From the peptide mass fingerprints which comprised about 99% of the WX-G250 sequence no O-linked glycosylation sites were detected.

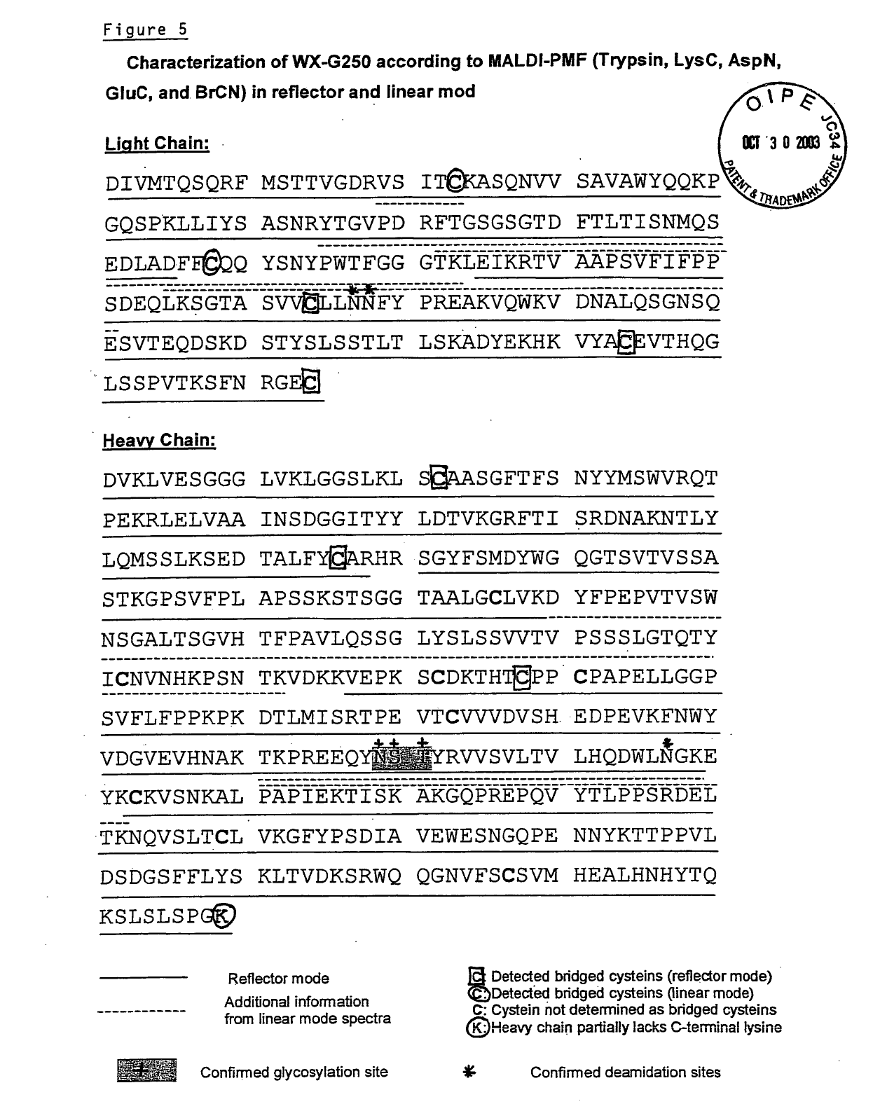

A summary of the results can be taken from FIG. 5.

LC-MS and LC-MS/MS of Tryptic Digest of cG250

FIG. 6 shows the sequence coverage of WX-G250 in the LC-MS/MS experiment of a tryptic digest without reduction and alkylation of the antibody.

The underlined sequences were detected as tryptic peptides. Detected disulfide bonds are marked. Cysteine residues undetected in this experiment are bold and underlined.

Edman Sequencing

To verify the posttranslational modifications fractions 5 and 33 of the trypsin digest and fractions 17 and 21 of the LysC digest were analyzed by automated Edman sequencing.

Tryptic fraction 5 contained the expected sequence E E Q Y ? corresponding to residues 295-298 (hc). The glycosylated N following the Y cannot be seen in Edman sequencing. Together with the peptide mass for peptide 295-303 determined by MALDI-MS it could be proven that this sequence was indeed glycosylated at position 300. Two minor contaminations were also found in this HPLC fraction: VSITC* and LIVSL.

VSITC* was derived from a Light chain peptide starting at position 19. It contained a Cys modified by iodacetamide. LIVSL could not be annotated to the WX-G250 structure. It is possible that this peptide was derived from trypsin.

Tryptic fraction 33 was not successful. No sequence could be determined, probably due to the limitation in sample amount (>>1 pmol).

LysC fraction 17 was close to the detection limit (<0.5 pmol) but proved to be the expected sequence: S? G? T? A S V V? C? L L?. However, due to limited amount of sample it was not possible to sequence to the expected deamidation site which followed the two leucins. But together with the MALDI-MS data the deamidation is evident.

LysC fraction 21 clearly showed the expected sequence T K P R E corresponding to residues 291-295 (hc). Together with the peptide mass for peptide 291-319 determined by MALDI-MS it could be proven that this sequence was indeed glycosylated at position 300. This is in accordance with the Edman sequencing result of tryptic fraction 5.

Fraction 23 of Lys C digest was analyzed by Edman sequencing to prove the identity of this very long fragment 150-202 (hc) which could only be detected in linear mode MALDI-MS. However, due to the small amount of sample it was impossible to get sequence information from this sample.

Conclusion

Sequence Verification

86% of the sequence of G250 was covered by peptide mass fingerprints using four different enzymes: trypsin, LysC, AspN, and GluC. Additional measurements in MALDI-MS linear mode increased the sequence coverage up to 99%. No deviations or mutations from the theoretically expected sequence were determined. However, one sequence heterogeneity (C-terminal lysine) was identified by peptide mass fingerprinting in reflector mode.

Verification of the Configuration of Disulfide Bonds

After peptide mass fingerprinting measured in reflector mode and linear mode MALDI mass spectrometry, respectively, four disulfide bridges (out of ten) were clearly determined. In Light chain disulfide bridges between Cys23-Cys88 and Cys-134-Cys194 were detected. In Heavy chain a disulfide bridge between Cys22-Cys96 was detected. Further, one disulfide bond connecting one Light and one Heavy chain could be identified: it was located at Cys214 (lc)-Cys222 (hc).

A summary of these results is shown in FIG. 4.

1. Recombinant vector system comprising at least one copy of a nucleic acid encoding the antigen-binding site of the heavy chain of an antibody comprising a nucleotide sequence encoding the CDR3 region (designated H3), or/and encoding the CDR2 region (designated H2), or/and encoding the CDR1 region (designated H1), as shown in FIG. 1 or/and FIG. 6, and at least one copy of a nucleic acid encoding the antigen-binding site of the light chain of an antibody comprising a nucleotide sequence encoding the CDR3 region (designated L3), or/and encoding the CDR2 region (designated L2), or/and encoding the CDR1 region (designated L1), as shown in FIG. 1 or/and FIG. 6, wherein the nucleic acid encoding the antigen-binding site of the heavy chain and of the light chain have separate expression control sequences.

2. Recombinant vector system according to claim 1 comprising a first recombinant vector comprising at least one copy of a nucleic acid encoding the antigen-binding site of the heavy chain and a second recombinant vector comprising at least one copy of a nucleic acid encoding the antigen-binding site of the light chain.

3. Recombinant vector system according to claim 1 wherein at least one copy of the nucleic acid encoding the antigen-binding site of the heavy chain and of the light chain are located on the same recombinant vector.

4. Method for the recombinant production of a polypeptide having an antigen-binding site comprising:
(a) providing a recombinant vector system according to claim 1,
(b) introducing the recombinant vector system into a suitable host cell,
(c) culturing the host cell under suitable conditions in a medium whereby an expression of the polypeptide takes place and
(d) obtaining the expressed product from the medium and/or the host cell.

5. The method of claim 4, wherein the host cell is a mammalian cell.

6. The method of claim 4, wherein between steps (a) and (b) a modification of the vector system takes place wherein the modification substantially does not alter the amino acid sequence of the antigen-binding site of the polypeptide to be expressed.

7. The method of claim 4 further comprising preparing a diagnostic or therapeutic agent from the expressed product.

8. The method of claim 7, wherein the expressed product is coupled to a diagnostic marker.

9. The method of claim 7, wherein the expressed product is coupled to a cytotoxic agent.

10. The method of claim 4, wherein the expressed product is selected from antibodies and antibody fragments.
